(19) United States
(12) Patent Application Publication
(10) Pub. No.: US 2021/0001796 A1
(43) Pub. Date: Jan. 7, 2021

(54) PHYSIQUE ESTIMATION DEVICE AND PHYSIQUE ESTIMATION METHOD

(71) Applicant: MITSUBISHI ELECTRIC CORPORATION, Tokyo (JP)

(72) Inventors: Daiki KUDO, Tokyo (JP); Takahiro OTSUKA, Tokyo (JP)

(73) Assignee: MITSUBISHI ELECTRIC CORPORATION, Tokyo (JP)

(21) Appl. No.: 17/023,562

(22) Filed: Sep. 17, 2020

Related U.S. Application Data
(63) Continuation of application No. PCT/JP2018/011415, filed on Mar. 22, 2018.

(51) Int. Cl. B60R 21/015

(52) U.S. Cl. CPC B60R 21/01552; B60R 21/01538; B60R 2021/006; B60R 21/01556; B60R 21/01554

(57) ABSTRACT

A physique estimation device determines a seat on which an occupant is seated on the basis of face information of the occupant detected from an image capturing a vehicle compartment, calculates the occupant's sitting height using a difference between a reference position and a face position for the determined seat, and estimates the occupant's physique on the basis of the occupant's sitting height.

### CROSS REFERENCE TO RELATED APPLICATIONS

This application is a Continuation of PCT International Application No. PCT/JP2018/011415, filed on Mar. 22, 2018, which is hereby expressly incorporated by reference into the present application.

### TECHNICAL FIELD

The present invention relates to a physique estimation device and a physique estimation method for estimating the physique of an occupant of a vehicle.

### BACKGROUND ART

Automobiles are mounted with airbags in order to prevent or mitigate injury to an occupant when an accident occurs. An airbag control device changes whether airbags can operate depending on the presence or absence of an occupant when an automobile accident occurs, and changes the pressure (unfolding expansion force) during activation of the airbag depending on the occupant's physique.

As conventional technology for estimating an occupant's physique in an automobile, for example, Patent Literature 1 describes a system that estimates an occupant's physique on the basis of an image taken by a stereo camera mounted on the automobile.

### CITATION LIST

### Patent Literature

Patent Literature 1: JP 2008-2838 A

### SUMMARY OF INVENTION

### Technical Problem

A stereo camera is more expensive than a monocular camera and requires a larger amount of calculation for image processing. For this reason, the system described in Patent Literature 1 has a disadvantage in that it requires an expensive computing device with high calculation performance.

The present invention solves the above disadvantage, and an object of the invention is to obtain a physique estimation device and a physique estimation method which can estimate the physique of an occupant of a vehicle using an image photographed by a monocular camera.

### Solution to Problem

A physique estimation device according to the present invention includes processing circuitry to receive input of an image capturing a vehicle compartment and detect face information of an occupant from the input image; and to estimate the occupant's physique by determining a seat on which the occupant is seated on the basis of the face information detected, calculating the occupant's sitting height using a difference between a reference position and a face position for the determined seat, and estimating the occupant's physique on the basis of the calculated occupant's sitting height. The processing circuitry determines the seat on which the occupant is seated on the basis of the face position and a face size of the occupant that are detected from the image capturing the vehicle compartment, and converts the difference between the reference position and the face position into the occupant's sitting height using a conversion value.

### Advantageous Effects of Invention

According to the invention, the physique estimation device determines a seat on which an occupant is seated on the basis of face information of the occupant detected from an image capturing the vehicle compartment, calculates the occupant's sitting height using a difference between a reference position and a face position for the determined seat, and estimates the occupant's physique on the basis of the occupant's sitting height.

Since it is only necessary to specify the face position from the face information of the occupant detected from the image, the image from which the face information is detected may be photographed by a monocular camera. This allows the physique estimation device to estimate the physique of the occupant of the vehicle using an image photographed by a monocular camera.

### BRIEF DESCRIPTION OF DRAWINGS

FIG. 1 is a block diagram illustrating an exemplary configuration of a physique estimation device according to a first embodiment of the invention.

FIG. 2 is a flowchart illustrating a physique estimation method according to the first embodiment.

FIG. 3 is a flowchart illustrating details of a physique estimation process in the first embodiment.

FIG. 4 is a diagram illustrating determination areas for respective seats in an image capturing a vehicle compartment.

FIG. 5A is a block diagram illustrating a hardware configuration for implementing functions of the physique estimation device according to the first embodiment. FIG. 5B is a block diagram illustrating a hardware configuration for executing software that implements functions of the physique estimation device according to the first embodiment.

FIG. 6 is a block diagram illustrating an exemplary configuration of a physique estimation device according to a second embodiment of the invention.

FIG. 7 is a flowchart illustrating details of a physique estimation process in the second embodiment.

FIG. 8 is a block diagram illustrating an exemplary configuration of a physique estimation device according to a third embodiment of the invention.

FIG. 9 is a flowchart illustrating details of a physique estimation process in the third embodiment.

FIG. 10 is a diagram illustrating an outline of calculation of shoulder width.

FIG. 11 is a diagram illustrating change in the position and the size of a face, the position and size corresponding to a slide position of a seat.

### DESCRIPTION OF EMBODIMENTS

To describe the present invention further in detail, embodiments for carrying out the invention will be described below with reference to the accompanying drawings.

### First Embodiment

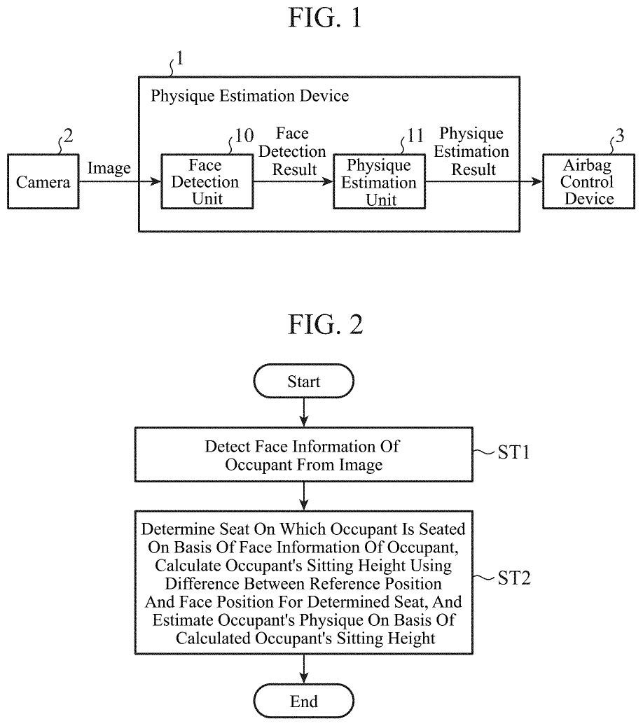

FIG. 1 is a block diagram illustrating an exemplary configuration of a physique estimation device 1 according to a first embodiment of the invention. The physique estimation device 1 receives input of an image of a vehicle compartment photographed by a camera 2, detects face information of an occupant from the input image, and determines the seat on which the occupant is seated on the basis of the detected face information. Then, the physique estimation device 1 calculates the occupant's sitting height using a difference between a reference position and a face position for the determined seat, and estimates the occupant's physique on the basis of the calculated occupant's sitting height.

In the first embodiment, it is assumed that the physique estimation device 1 is an onboard device; however, the physique estimation device 1 may be provided outside the vehicle. For example, the physique estimation device 1 may be implemented by a server device external to the vehicle. In this case, the server device receives a photographed image of the vehicle compartment from an onboard communication device, and sends a physique estimation result of an occupant estimated on the basis of the received photographed image to the onboard communication device. The onboard communication device outputs the received physique estimation result of the occupant to an airbag control device 3.

The camera 2 is a monocular camera an imaging range of which ranges over the vehicle compartment. For example, the camera 2 may be an infrared camera capable of photographing in a dark place. Alternatively, the camera 2 may be a visible light region camera, such as a CCD camera and a CMOS camera.

The number and mounting position of at least one camera 2 are adjusted so that all the occupants in the vehicle compartment are captured. For example, a single camera 2 mounted in the vicinity of a rearview mirror in the vehicle compartment can capture a wide region in the vehicle compartment. Note that two or more cameras 2 may be provided. For example, a camera 2 may be provided for each seat to include the corresponding seat in its imaging range.

A physique estimation result of an occupant obtained by the physique estimation device 1 for each occupied position is output to the airbag control device 3. The airbag control device 3 controls the activation of the airbags on the basis of the occupant's physique estimation results input from the physique estimation device 1. For example, the airbag control device 3 changes the pressure during activation of the airbag depending on the occupant's physique.

The physique estimation device 1 includes a face detection unit 10 and a physique estimation unit 11.

The face detection unit 10 receives input of an image capturing the vehicle compartment and detects face information of an occupant from the input image. The physique estimation unit 11 determines the seat on which the occupant is seated on the basis of the face information detected by the face detection unit 10, calculates the occupant's sitting height using a difference between a reference position and a face position for the determined seat, and estimates the occupant's physique on the basis of the calculated occupant's sitting height.

Next, the operation will be described.

FIG. 2 is a flowchart illustrating a physique estimation method according to the first embodiment, and illustrates a series of processes from the input of an image capturing the vehicle compartment to estimation of the occupant's physique.

The face detection unit 10 receives input of an image capturing the vehicle compartment from the camera 2 and detects face information of the occupant from the input image (step ST1). For the detection process of the face information, for example, an image recognition method using Haar-like features may be used, or other known image recognition methods may be used. Moreover, the face detection unit 10 may narrow down the face detection target to determination areas for occupied positions described later, instead of setting the entire image as the face detection target. Thereby, the amount of calculation required for face detection can be reduced.

Face information is information indicating a face area in an image, and includes coordinates indicating the position and the size of the face. The face area may be an area of the entire face, but may alternatively be a partial area of the face as long as the position and the size of the face can be specified.

The coordinates indicating the position and the size of the face may be, for example, the coordinates of two diagonal points on a rectangle in which the face area is inscribed. The two diagonal points are, for example, the upper left point and the lower right point on the rectangle. Of points on the rectangle, two points in the width direction of the face and/or two points in the height direction may be used. The face information may include coordinates of face parts (eyes, nose, mouth, ears). The face detection unit 10 outputs a face detection result including these pieces of face information to the physique estimation unit 11.

Next, the physique estimation unit 11 determines a seat on which the occupant is seated on the basis of the face information of the occupant, calculates the occupant's sitting height using a difference between a reference position and a face position for the determined seat, and estimates the occupant's physique on the basis of the calculated occupant's sitting height (step ST2).

For example, the physique estimation unit 11 specifies the position and the size of the occupant's face from the face information detected by the face detection unit 10, and determines the seat on which the occupant is seated on the basis of the specified position and size of the face. The physique estimation unit 11 converts the difference between the reference position and the face position into the occupant's sitting height using a conversion value corresponding to the determined seat. On the basis of the occupant's sitting height obtained by converting the difference, the physique estimation unit 11 refers to correspondence data between anthropometric data including the sitting height and human physiques, and thereby estimates the occupant's physique corresponding to the sitting height value.

Next, details of a physique estimation process will be described.

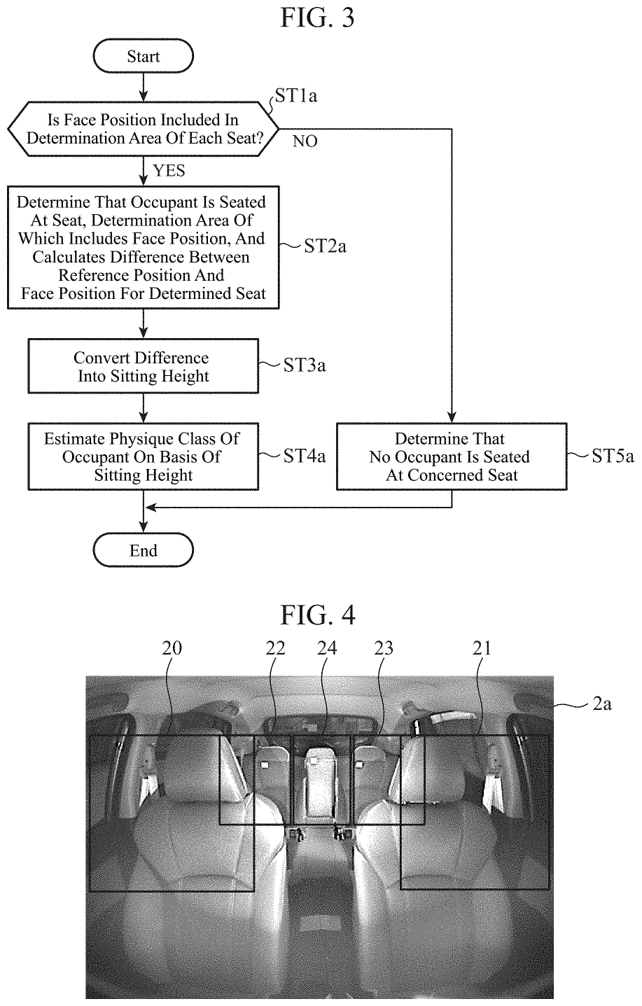

FIG. 3 is a flowchart illustrating details of the physique estimation process in the first embodiment, and illustrates specific processes of step ST2 of FIG. 2.

In step ST1a, the physique estimation unit 11 determines the seat on which the occupant is seated from the face detection result having been input from the face detection unit 10. For example, the physique estimation unit 11 determines whether the position of a face is included in a determination area for each seat on the basis of coordinates of the position of the face specified from the face detection result.

FIG. 4 is a diagram illustrating determination areas for respective seats in an image 2a capturing a vehicle compartment.

The image 2a is obtained by the camera 2 capturing the vehicle compartment. Each of the determination areas 20 to 24 is set for the corresponding seat and indicates an image area in which a face is located when an occupant is seated. The determination areas 20 to 24 are determined in advance by experiments.

Note that each of the determination areas 20 to 24 is an image area adjusted to include the face of an occupant not limited to when the occupant seated in a corresponding seat is facing the front, but also when the occupant is facing sideways or when the occupant is facing down or up.

The determination area 20 is an image area in which the face of an occupant seated in the driver's seat is located, and the determination area 21 is an image area in which the face of an occupant seated in the passenger seat is located. The determination area 22 is an image area in which the face of an occupant seated in the rear seat behind the driver's seat is located, and the determination area 23 is an image area in which the face of an occupant seated in the rear seat behind the passenger seat is located. The determination area 24 is an image area in which the face of an occupant seated in the center rear seat is located.

The physique estimation unit 11 determines a seat on which the occupant is seated on the basis of the face size specified from the face detection result when the face position is included in multiple determination areas among the determination areas 20 to 24. For example, the determination area 20 corresponding to the driver's seat and the determination area 22 corresponding to the rear seat behind the driver's seat partially overlap as illustrated in FIG. 4. When the physique estimation unit 11 determines that the position of the face is included in the overlapped area, the face size is specified from the face detection result, and if the specified face size is greater than a threshold value, it is determined that the occupant is seated in the driver's seat (seat closer to the camera 2). If the face size is equal to or less than the threshold value, the physique estimation unit 11 determines that the occupant is seated in the rear seat (seat on a far side from the camera 2). Note that the threshold value used for determination as to the face size is determined in advance by experiments.

Let us return to the explanation of FIG. 3.

If it is determined that the face position is included in the determination area (step ST1a: YES), the physique estimation unit 11 determines that an occupant is seated in the seat corresponding to the determination area, and calculates the difference between the reference position and the face position for the determined seat (step ST2a). A position in the face used for calculation of the difference may be any of the upper end position, the lower and position, and the center position of the face area, or may be the position of any face part (eyes, nose, mouth, or ears).

The reference position is, for example, the height position of the seat surface, and is represented by coordinate values of a two-dimensional coordinate system of the image photographed by the camera 2. In a case where the seat surface is captured in the image, coordinates of the height position of the seat surface in the image are set as the reference position. In a case where the seat surface is not captured in the image, coordinates of a position in a two-dimensional coordinate system obtained by extending the two-dimensional coordinate system of the image to the outside of the image, the position being assumed to be the height position of the seat surface, are set as the reference position. The reference position is determined in advance by experiments.

Next, the physique estimation unit 11 converts the difference between the reference position and the face position into the occupant's sitting height using a conversion value (step ST3a). The conversion value is a value for converting a distance between points in the two-dimensional coordinate system of the image into a distance in the vertical direction in the real space. The conversion value is determined in advance by experiments.

The reference position and the conversion value may be varied for each seat, or may be shared by the seats. For example, the height of the seat surface of the driver's seat and the height of the surface of the passenger seat are generally the same, and thus a shared reference value is used, and also the heights of the three rear seats illustrated in FIG. 4 are the same, and thus a shared reference value is used.

The physique estimation unit 11 estimates the physique class of the occupant on the basis of the occupant's sitting height (step ST4a). For example, the physique estimation unit 11 compares the occupant's sitting height calculated in step ST3a with a classification threshold value, and estimates the occupant's physique class in accordance with the comparison result. Physique classes are classes for classifying the size of an occupant, and are determined, for example, in accordance with a criterion for changing the pressure of an airbag. The threshold value for the classification of physique classes is determined in advance by experiments.

The physique estimation result (for example, physique class) of the occupant by the physique estimation unit 11 is output from the physique estimation device 1 to the airbag control device 3. The airbag control device 3 changes the pressure during activation of the airbag on the basis of the physique estimation result of the occupant input from the physique estimation device 1.

Note that if it is determined that the position of the face is not included in the determination area (step ST1a: NO), the physique estimation unit 11 determines that no occupant is seated in the seat corresponding to the determination area (step ST5a). Thereafter, the physique estimation unit 11 terminates the process related to the seat.

A sitting height is a distance from a seat surface to the top of the head of a person. In the first embodiment, however, there are cases as described above in which, for example, coordinates of the lower end position of the face or coordinates of the center position of the face are detected as the coordinates that indicate the face position. In these cases, in a case where the coordinates of the lower end position of the face are detected as the coordinates of the face position, the physique estimation unit 11 calculates the distance from the seat surface to the lower end position of the face, and calculates the sitting height by adding, to the calculated value, a standard value of length of a face. Likewise, in a case where the coordinates of the center position of the face are detected as the coordinates of the face position, the physique estimation unit 11 calculates the distance from the seat surface to the center position of the face, and calculates the sitting height by adding, to the calculated value, a value obtained by multiplying the standard value of length of a face by ½.

Hereinafter, hardware configurations for implementing the physique estimation device 1 will be described.

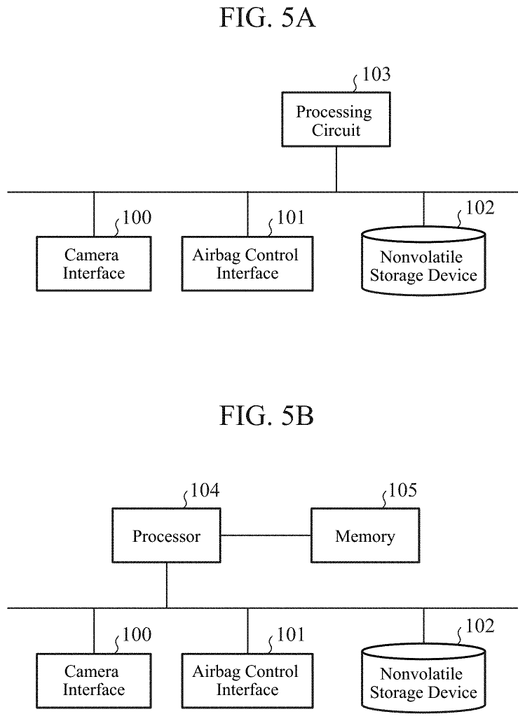

FIG. 5A is a block diagram illustrating a hardware configuration for implementing the functions of the physique estimation device 1. FIG. 5B is a block diagram illustrating a hardware configuration for executing software that implements the function of the physique estimation device 1. In FIG. 5A and FIG. 5B, a camera interface 100 is an interface between the physique estimation device 1 and the camera 2 illustrated in FIG. 1, and relays image information output from the camera 2 to the physique estimation device 1. An airbag control interface 101 is an interface between the physique estimation device 1 and the airbag control device 3 illustrated in FIG. 1, and relays the physique estimation result output from the physique estimation device 1 to the airbag control device 3.

A nonvolatile storage device 102 stores information obtained by the physique estimation process on the occupant performed by the physique estimation device 1. The nonvolatile storage device 102 stores image information input from the camera 2, face information detected by the face detection unit 10, threshold value information used for various determinations, reference values, conversion values, and physique estimation results. The nonvolatile storage device 102 may be provided independently of the physique estimation device 1. For example, a storage device on the cloud may be used as the nonvolatile storage device 102.

The functions of the face detection unit 10 and the physique estimation unit 11 in the physique estimation device 1 are implemented by a processing circuit. More specifically, the physique estimation device 1 includes a processing circuit for executing the processes from step ST1 to step ST2 illustrated in FIG. 2. The processing circuit may be dedicated hardware or a central processing unit (CPU) for executing a program stored in a memory.

In the case where the processing circuit is a processing circuit 103 of dedicated hardware illustrated in FIG. 5A, the processing circuit 103 may be a single circuit, a composite circuit, a programmed processor, a parallel programmed processor, an application specific integrated circuit (ASIC), a field-programmable gate array (FPGA), or a combination thereof, for example. The functions of the face detection unit 10 and the physique estimation unit 11 may be implemented by separate processing circuits, or may be collectively implemented by a single processing circuit.

In the case where the processing circuit is a processor 104 illustrated in FIG. 5B, the functions of the face detection unit 10 and the physique estimation unit 11 are implemented by software, firmware, or a combination of software and firmware. The software or the firmware is described as a program and is stored in a memory 105.

The processor 104 reads and executes the program stored in the memory 105 and thereby implements functions of the face detection unit 10 and the physique estimation unit 11. More specifically, the physique estimation device 1 includes the memory 105 for storing programs execution of which by the processor 104 results in execution of the processes from step ST1 to step ST2 illustrated in FIG. 2. These programs cause a computer to execute procedures or methods of the face detection unit 10 and the physique estimation unit 11. The memory 105 may be a computer-readable storage medium storing the programs for causing a computer to function as the face detection unit 10 and the physique estimation unit 11.

The memory 105 corresponds to, for example, a nonvolatile or volatile semiconductor memory, such as a random access memory (RAM), a read only memory (ROM), a flash memory, an erasable programmable read only memory (EPROM), and an electrically-EPROM (EEPROM), a magnetic disc, a flexible disc, an optical disc, a compact disc, a mini disc, or a DVD.

Furthermore, a part of the functions of the face detection unit 10 and the physique estimation unit 11 may be implemented by dedicated hardware, and another part thereof may be implemented by software or firmware. For example, the function of the face detection unit 10 is implemented by a processing circuit as dedicated hardware. The function of the physique estimation unit 11 may be implemented by the processor 104 reading and executing programs stored in the memory 105. In this manner, the processing circuit can implement the functions described above by hardware, software, firmware, or a combination thereof.

As described above, the physique estimation device 1 according to the first embodiment determines a seat on which an occupant is seated on the basis of face information of the occupant detected from an image, calculates the occupant's sitting height using a difference between a reference position and a face position for the determined seat, and estimates the occupant's physique on the basis of the occupant's sitting height. In particular, the physique estimation unit 11 determines the seat on which the occupant is seated on the basis of the position and the size of the face of the occupant that are detected from the image, and converts the difference between the reference position and the face position into the occupant's sitting height using a conversion value corresponding to the determined seat. Since it is only necessary to specify the face position from the face information of the occupant detected from the image, the image from which the face information is detected may be photographed by a monocular camera. This allows the physique estimation device 1 to estimate the physique of the occupant of the vehicle using an image photographed by a monocular camera.

### Second Embodiment

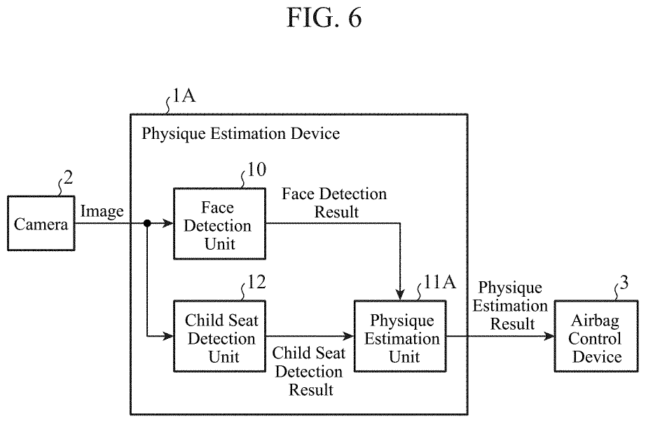

FIG. 6 is a block diagram illustrating a configuration of a physique estimation device 1A according to a second embodiment of the invention. In FIG. 6, the same component as that in FIG. 1 is denoted by the same symbol and descriptions thereof are omitted. The physique estimation device 1A estimates the physique of an occupant like in the first embodiment and also estimates that the physique of an occupant seated on a child seat is a physique that is classified as that of a child without performing the physique estimation process described in the first embodiment when the child seat is detected.

The physique estimation device 1A includes a face detection unit 10, a physique estimation unit 11A, and a child seat detection unit 12. Like in the first embodiment, the physique estimation unit 11A determines a seat on which an occupant is seated on the basis of face information detected by the face detection unit 10, calculates the occupant's sitting height using a difference between a reference position and a face position for the determined seat, and estimates the occupant's physique on the basis of the occupant's sitting height. The physique estimation unit 11A further determines an occupant seated on the child seat on the basis of a child seat detection result input from the child seat detection unit 12, and estimates that the physique of the determined occupant is a physique classified as that of a child.

The child seat detection unit 12 receives input of an image obtained by a camera 2 capturing the vehicle compartment and detects child seat information from the input image. For a detection process of the child seat information, for example, an image recognition method using histogram of oriented gradients (HOG) may be used, or other known image recognition methods may be used. The child seat detection result includes, for example, position coordinates of the child seat in the image. Moreover, the position coordinates of the child seat may be, for example, position coordinates of a point included in the child seat area in the image, or may be position coordinates of a point on a rectangle in which this area is inscribed.

A processing circuit that implements the functions of the face detection unit 10, the physique estimation unit 11A, and the child seat detection unit 12 may be the processing circuit 103 that is dedicated hardware illustrated in FIG. 5A. Alternatively, the processing circuit that implements the functions of the face detection unit 10, the physique estimation unit 11A, and the child seat detection unit 12 may be the processor 104 that executes programs stored in the memory 105 illustrated in FIG. 5B.

Further alternatively, some of the functions of the face detection unit 10, the physique estimation unit 11A, and the child seat detection unit 12 may be implemented by dedicated hardware, and others may be implemented by software or firmware.

Next, the operation will be described.

Since the operation of the face detection unit 10 is similar to that of the first embodiment, description thereof is omitted.

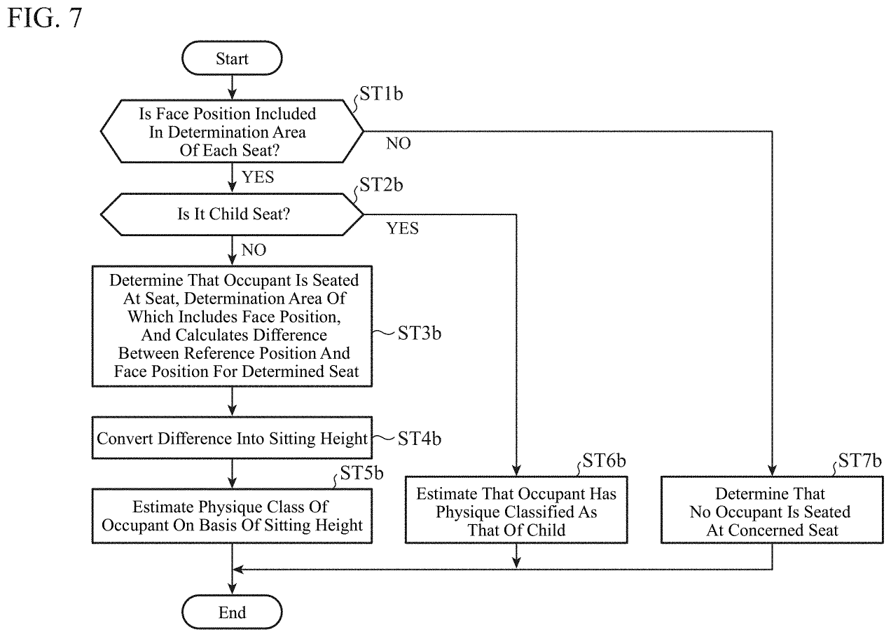

FIG. 7 is a flowchart illustrating details of a physique estimation process in the second embodiment. The process of step ST1b, the processes from step ST3b to step ST5b, and the process of step ST7b in FIG. 7 are the same as the processes from step ST1a to step ST5a illustrated in FIG. 3, and thus description thereof is omitted.

If it is determined that the position of a face is included in a determination area (step ST1b: YES), the physique estimation unit 11A determines that an occupant is seated in a seat corresponding to the determination area.

Subsequently, the physique estimation unit 11A determines whether the seat on which the occupant is seated is a child seat on the basis of the child seat detection result input from the child seat detection unit 12 (step ST2b). For example, in a case where the position coordinates of the child seat are included in an image area of the seat determined to be the one on which the occupant is seated, the physique estimation unit 11A determines that the seat determined to be the one on which the occupant is seated is the child seat.

If it is determined that the seat on which the occupant is seated is not a child seat (step ST2b: NO), the physique estimation unit 11A executes a series of processes from step ST3b.

If no child seat information is detected from the image by the child seat detection unit 12, the physique estimation unit 11A determines that none of the seats on which occupants are seated are child seats, and thus, likewise, a series of processes from step ST3b are executed.

If it is determined that the seat on which the occupant is seated is a child seat (step ST2b: YES), the physique estimation unit 11A estimates that the physique of the occupant seated on this seat is a physique classified as that of a child (step ST6b). The physique estimation result of the occupant by the physique estimation unit 11A is output from the physique estimation device 1A to an airbag control device 3. The airbag control device 3 changes the pressure (unfolding expansion force) of the airbag for the child seat to a pressure that is suitable for a child.

As described above, the physique estimation device 1A according to the second embodiment includes the child seat detection unit 12. The physique estimation unit 11A determines an occupant seated on a child seat on the basis of child seat information detected by the child seat detection unit 12, and estimates that the physique of the determined occupant is a physique classified as that of a child. The physique estimation unit 11A estimates that the physique of the occupant seated on the child seat is a physique classified as that of a child without calculating the occupant's sitting height. Thereby, the amount of calculation required for the physique estimation process can be reduced.

### Third Embodiment

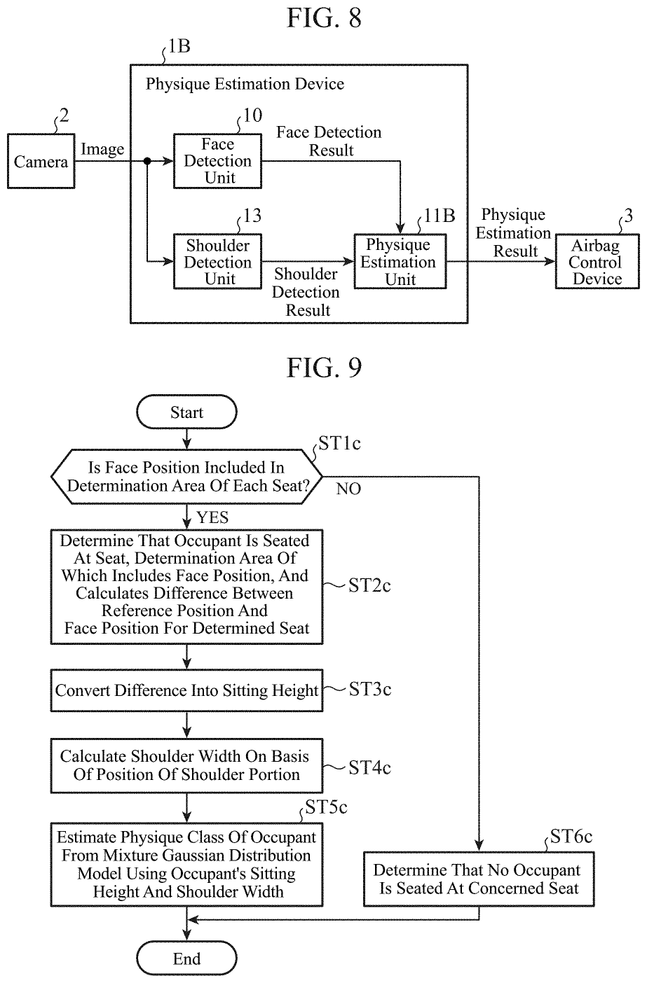

FIG. 8 is a block diagram illustrating a configuration of a physique estimation device 1B according to a third embodiment of the invention. In FIG. 8, the same component as that in FIG. 1 is denoted by the same symbol and descriptions thereof are omitted. The physique estimation device 1B detects face information and shoulder information of an occupant from an image of a vehicle compartment, and determines a seat on which the occupant is seated on the basis of the detected face information. The physique estimation device 1B calculates the occupant's sitting height from the seat on which the occupant is seated and the face information, calculates the shoulder width of the occupant from the shoulder information, and estimates the occupant's physique on the basis of the shoulder width and the sitting height of the occupant.

The physique estimation device 1B includes a face detection unit 10, a physique estimation unit 11B, and a shoulder detection unit 13.

Like in the first embodiment, the physique estimation unit 11B determines a seat on which an occupant is seated on the basis of face information detected by the face detection unit 10, and calculates the occupant's sitting height using a difference between a reference position and a face position for the determined seat. Furthermore, the physique estimation unit 11B calculates the occupant's shoulder width on the basis of a shoulder detection result by the shoulder detection unit 13, and estimates the occupant's physique on the basis of the shoulder width and the sitting height of the occupant.

The shoulder detection unit 13 receives input of an image capturing the vehicle compartment and detects shoulder information of the occupant from the input image. For the detection process of the shoulder information, for example, an image recognition method using HOG may be used. Alternatively, a shoulder area may be detected from the image by template matching using templates of shoulder images. However, other known image recognition methods may be used.

The shoulder detection result includes, for example, position coordinates of the shoulder portion in the image. The position coordinates of the shoulder portion may be position coordinates of a point included in the shoulder portion area in the image, or may be position coordinates of a point on a rectangle in which the shoulder portion area is inscribed.

For example, there are cases in which the right shoulder of an occupant seated in a rear seat behind a driver's seat in a right-hand drive vehicle is not captured by a camera 2 being blocked by the driver's seat. In this case, the shoulder detection unit 13 can detect only shoulder information of the left shoulder of the occupant. There are also cases in which the left shoulder of an occupant seated in a rear seat behind a passenger seat is not captured by the camera 2 being blocked by the passenger seat. In this case, the shoulder detection unit 13 can detect only shoulder information of the right shoulder of the occupant.

Therefore, the shoulder detection unit 13 may detect the shoulder area by separately using an image feature amount for the left shoulder and an image feature amount for the right shoulder depending on the seat. The shoulder detection unit 13 may detect the shoulder area also by separately using templates for the left shoulder and the right shoulder depending on the seat.

A processing circuit that implements the functions of the face detection unit 10, the physique estimation unit 11B, and the shoulder detection unit 13 may be the processing circuit 103 that is dedicated hardware illustrated in FIG. 5A. Alternatively, the processing circuit that implements the functions of the face detection unit 10, the physique estimation unit 11B, and the shoulder detection unit 13 may be the processor 104 that executes programs stored in the memory 105 illustrated in FIG. 5B. A part of the functions of the face detection unit 10, the physique estimation unit 11B, and the shoulder detection unit 13 may be implemented by dedicated hardware, and another part thereof may be implemented by software or firmware.

Next, the operation will be described.

Since the operation of the face detection unit 10 is similar to that of the first embodiment, description thereof is omitted.

FIG. 9 is a flowchart illustrating details of a physique estimation process in the third embodiment. The processes from step ST1c to step ST3c, and the process of step ST6c in FIG. 9 are the same as the processes from step ST1a to step ST3a and the process of step ST5a illustrated in FIG. 3, and thus description thereof is omitted.

The physique estimation unit 11B calculates the shoulder width of an occupant on the basis of the position of a shoulder portion included in a shoulder detection result (step ST4c). For example, the physique estimation unit 11B calculates the difference between the position coordinates of the left and right shoulder portions, and converts the difference into the shoulder width using a conversion value when the position coordinates of both the left and right shoulder portions are obtained. The conversion value is a value for converting a distance between points in the two-dimensional coordinate system of the image into a distance in the horizontal direction in the real space. The conversion value is determined in advance by experiments.

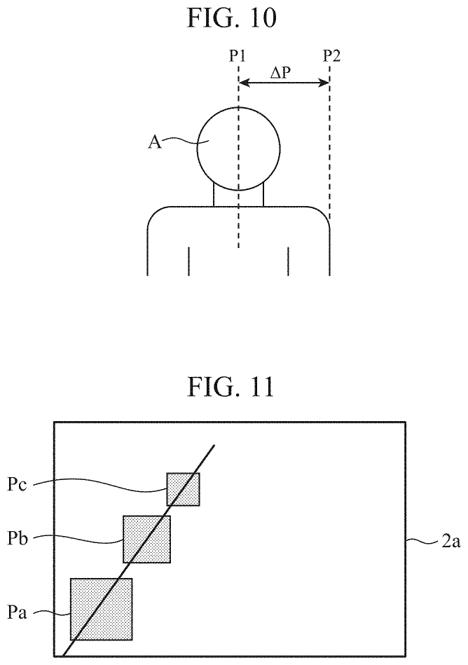

FIG. 10 is a diagram illustrating an outline of calculation of the shoulder width. As illustrated in FIG. 10, the physique estimation unit 11B calculates a coordinate P1 of the center position of the face of an occupant A from a face detection result, and calculates a coordinate P2 of an end position of a shoulder of the occupant A from a shoulder detection result. In a case where only one of the shoulders of the occupant A is detected by the shoulder detection unit 13, the physique estimation unit 11B calculates a difference ΔP between the coordinate P1 of the center position of the face and the coordinate P2 of the end position of the shoulder, and converts a value obtained by multiplying this difference ΔP by 2 into the shoulder width.

Next, the physique estimation unit 11B estimates the physique class of the occupant from a mixture Gaussian distribution model using the occupant's sitting height and shoulder width (step ST5c).

For example, the physique estimation unit 11B acquires mixture Gaussian distribution parameters for four mixture components (adult male, adult female, minor male, minor female) on the basis of the weight, the sitting height, and the shoulder width of people included in anthropometric statistics data. Subsequently, the physique estimation unit 11B estimates the physique class of the occupant from the mixture Gaussian distribution model defined by the mixture Gaussian distribution parameters, using the sitting height and the shoulder width having been obtained from the image. The mixture Gaussian distribution model is the correspondence data between anthropometric data including the sitting height and the shoulder width and human physiques.

Although the mixture Gaussian distribution is used for estimation of the physique, the physique estimation unit 11B may use a support vector machine (SVM), which is a data clustering method, for estimation of the physique. Furthermore, parameters other than the body weight may be used as a reference for classification of the physique class, and the number of mixture components of the mixture Gaussian distribution parameters may be modified.

As described above, the physique estimation device 1B according to the third embodiment includes the shoulder detection unit 13. The physique estimation unit 11B calculates the shoulder width of the occupant on the basis of the shoulder information detected by the shoulder detection unit 13, and estimates the occupant's physique on the basis of the calculated shoulder width and sitting height of the occupant.

In particular, the physique estimation unit 11B estimates the occupant's physique from the shoulder width and sitting height of the occupant by referring to the correspondence data between anthropometric data including the sitting height and the shoulder width and human physiques.

In this manner, since it is only necessary to be able to specify the face position from face information of an occupant having been detected from an image and to specify the position of a shoulder from shoulder information, the image from which the face information and the shoulder information are to be detected may be photographed by a monocular camera. This allows the physique estimation device 1B to estimate the physique of the occupant of the vehicle using an image photographed by a monocular camera.

### Fourth Embodiment

In general, seats of a vehicle can be slid or reclined.

In a case where a camera 2 is facing to the rear side from the front of the vehicle, an occupant seated on a seat moves away from or approaches the camera 2 when the seat is slid or reclined. The face position of the occupant, moving away from the camera 2 by sliding or reclining the seat, moves upward in an image photographed by the camera 2. The face position of the occupant, approaching the camera 2 by sliding or reclining the seat, moves downward in the image. For this reason, the face position may deviate from the determination area due to sliding or reclining of the seat.

On the other hand, the face size of the occupant, moving away from the camera 2 by sliding or reclining the seat, becomes smaller in the image photographed by the camera 2.

Contrarily, the face size of the occupant, approaching the camera 2 by sliding or reclining the seat, becomes bigger in the image.

Therefore, the physique estimation unit according to any one of the first to third embodiments may determine a seat on which an occupant is seated by referring to data indicating face position ranges corresponding to respective slide positions or reclining positions of the seat and face size ranges corresponding to the respective slide positions or reclining positions.

FIG. 11 is a diagram illustrating change in the position of the face of an occupant, the position of the face corresponding to the slide position of a seat, and illustrates an image 2a in which a vehicle compartment is photographed by the camera 2 facing to the rear side from the front of the vehicle.

When the driver's seat is slid forward, the position of the face of the occupant seated in the driver's seat moves outward and downward in the image 2a, and the face size increases. When the driver's seat is slid backward, the position of the face of the occupant seated in the driver's seat moves toward the center and upward in the image 2a, and the face size is reduced.

In order to reflect change in the position and the size of the face in the image 2a accompanying sliding of the seat, determination areas Pa, Pb, and Pc, which are as illustrated in FIG. 11, corresponding to slide positions of the seat are set in the physique estimation unit. The determination area Pa indicates an image area in which the face of an occupant seated in the driver's seat that is slid the most forward is located. The determination area Pb indicates an image area in which the face of an occupant seated in the driver's seat that is slid to the normal position is located. The determination area Pc indicates an image area in which the face of an occupant seated in the driver's seat that is slid most backward is located.

Since the face size of the occupant seated in the driver's seat that is slid forward becomes large in the image 2a, the determination area Pa that is the widest image area is set in the physique estimation unit.

Since the face size of the occupant seated in the driver's seat that is slid backward becomes small in the image 2a, the determination area Pc that is the narrowest image area is set in the physique estimation unit.

An image area having an intermediate area and corresponding to the normal slide position of the driver's seat is the determination area Pb.

Note that the determination areas Pa, Pb, and Pc are determined in advance by experiments. The physique estimation unit determines that the occupant is seated in the driver's seat when the face position specified from a face detection result is included in one of the determination areas Pa, Pb, and Pc.

The case where the seat is slid has been described as an example in FIG. 11. Likewise in a case where the seat is reclined, determination areas that correspond to reclining positions are used for determination of the seat.

In addition, the reference position and conversion values used for calculation of the occupant's sitting height also have different values corresponding to slide positions or reclining positions of the seat.

Therefore, reference positions and conversion values that correspond to slide positions or reclining positions are set in the physique estimation unit. When having determined the seat on which an occupant is seated, the physique estimation unit calculates the sitting height of the occupant using the conversion value and the reference position that correspond to the slide position or the reclining position. This allows the physique estimation unit to accurately determine the seat on which the occupant is seated and to calculate the accurate sitting height even when the seat is slid or reclined.

Note that the present invention is not limited to the above embodiments, and the present invention may include a flexible combination of the individual embodiments, a modification of any component of the individual embodiments, or omission of any component in the individual embodiments within the scope of the present invention.

### INDUSTRIAL APPLICABILITY

A physique estimation device according to the present invention is capable of estimating the physique of an occupant of a vehicle using an image photographed by a monocular camera, and thus is applicable to control of airbags, for example.

### REFERENCE SIGNS LIST

1, 1A, 1B: physique estimation device, 2: camera, 2a: image, 3: airbag control device, 10: face detection unit, 11, 11A, 11B: physique estimation unit, 12: child seat detection unit, 13: shoulder detection unit, 20 to 24: determination area, 100: camera interface, 101: airbag control interface, 102: nonvolatile storage device, 103: processing circuit, 104: processor, 105: memory

1. A physique estimation device comprising:
processing circuitry
to receive input of an image capturing a vehicle compartment and detect face information of an occupant from the input image; and
to estimate the occupant's physique by determining a seat on which the occupant is seated on a basis of the face information detected, calculating the occupant's sitting height using a difference between a reference position and a face position for the determined seat, and estimating the occupant's physique on a basis of the calculated occupant's sitting height,
wherein the processing circuitry determines the seat on which the occupant is seated on a basis of the face position and a face size of the occupant that are detected from the image capturing the vehicle compartment, and converts the difference between the reference position and the face position into the occupant's sitting height using a conversion value.

2. A physique estimation device comprising:
processing circuitry
to receive input of an image capturing a vehicle compartment and detect face information of an occupant from the input image;
to estimate the occupant's physique by determining a seat on which the occupant is seated on a basis of the face information detected, calculating the occupant's sitting height using a difference between a reference position and a face position for the determined seat, and estimating the occupant's physique on a basis of the calculated occupant's sitting height; and
to receive input of the image capturing the vehicle compartment and detect child seat information from the input image,
wherein the processing circuitry identifies an occupant seated on a child seat on a basis of the child seat information detected, and estimates that a physique of the identified occupant is a physique classified as that of a child.

3. The physique estimation device according to claim 1,
wherein the processing circuitry receives input of the image capturing the vehicle compartment, and detects shoulder information of the occupant from the input image, and
the processing circuitry calculates a shoulder width of the occupant on a basis of the shoulder information detected, and estimates the occupant's physique on a basis of the calculated shoulder width and the sitting height of the occupant.

4. The physique estimation device according to claim 3,
wherein the processing circuitry estimates the occupant's physique from the shoulder width and the sitting height of the occupant by referring to correspondence data between anthropometric data including sitting heights and shoulder widths and human physiques.

5. The physique estimation device according to claim 1,
wherein the processing circuitry determines the seat on which the occupant is seated on a basis of the face information detected by referring to data indicating face position ranges corresponding to respective slide positions or reclining positions of the seat and face size ranges corresponding to the respective slide positions or reclining positions, and calculates the occupant's sitting height using the conversion value and the reference position that correspond to a slide position or a reclining position.

6. A physique estimation method comprising:
receiving input of an image capturing a vehicle compartment and detecting a face position and a face size of an occupant from the input image; and
estimating the occupant's physique by determining a seat on which the occupant is seated on a basis of the face position and the face size detected, calculating the occupant's sitting height by converting a difference between a reference position and a face position for the determined seat into the occupant's sitting height using a conversion value, and estimating the occupant's physique on a basis of the calculated occupant's sitting height.

7. A physique estimation method comprising:
receiving input of an image capturing a vehicle compartment and detecting face information of an occupant from the input image;
estimating the occupant's physique by determining a seat on which the occupant is seated on a basis of the face information detected, calculating the occupant's sitting height using a difference between a reference position and a face position for the determined seat, and estimating the occupant's physique on a basis of the calculated occupant's sitting height;
receiving input of the image capturing the vehicle compartment and detecting child seat information from the input image; and
identifying an occupant seated on a child seat on a basis of the child seat information detected and estimating that a physique of the identified occupant is a physique classified as that of a child.
